(19) United States
(12) Patent Application Publication
(10) Pub. No.: US 2005/0019258 A1
(43) Pub. Date: Jan. 27, 2005

(54) Compound testing method

(75) Inventors: Jennifer C. Gardiner, Sandwich (GB); Johannes F. Zanzinger, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/890,603

(22) Filed: Jul. 13, 2004

Related U.S. Application Data
(60) Provisional application No. 60/496,770, filed on Aug. 21, 2003.

(30) Foreign Application Priority Data
Jul. 15, 2003 (GB) 0316539.6

(51) Int. Cl. A61K 49/00

(52) U.S. Cl. 424/9.1

(57) ABSTRACT

This invention relates to a method for the evaluation of potential therapeutic agents for the treatment of incontinence, as well as to a method for discovering new theraprutic agents for the treatment of incontinence. The method is based on instilling cold liquid into the bladder of a guinea pig in the presence or absence of a test compound, and measuring the lowering of the threshold volume for bladder contractions compared to the normal cystometric capacity.

This application claims priority to GB Application Serial No. 0316539.6, filed Jul. 15, 2003 and U.S. Provisional Application Ser. No. 60/496,770, filed Aug. 21, 2003.

This invention relates to a method for the evaluation of potential therapeutic agents for the treatment of incontinence. This invention also relates to a method for discovering new therapeutic agents for the treatment of incontinence.

A positive ice-water test (IWT) following instillation of ice-cold water or saline into the bladder is characterised by involuntary sustained bladder contractions at a threshold volume lower than normal cystometric capacity. This bladder cooling reflex has been demonstrated in patients with spinal upper but not lower motor neurone lesions and in a large proportion of patients with incontinence associated with neurological disorders such as Parkinson's disease, cerebrovascular accident and multiple sclerosis (Bors and Blinn (1957) Arch. Neurol. Psychiatr. 78, 339-354; Geirsson, G. et al. (1993) Br. J. Urol. 71, 681-685; Geirsson G. et al. (1994) Br. J. Urol. 73, 498-503; Ishigooka et al. (1997) Urol. Int. 58, 84-87; Ronzoni et al. (1997) Br. J. Urol. 79, 698-701). Although a positive IWT has been reported not to be present in neurologically normal subjects (Geirsson, G. et al. (1993) Br. J. Urol. 71, 681-685; Ishigooka et al (1997) Urol. Int. 58, 84-87), the reflex was recorded in a majority of elderly patients with uninhibited overactive bladder who lacked a neurological diagnosis but in whom a specific, limited but undetected neuropathy could have existed (Geirsson, G et al. (1993) Age and Ageing 22, 125-131). Infants up to 4 years old also exhibit a positive IWT, this likely becoming suppressed in later years by a descending inhibitory pathway during maturation of the central nervous system (Geirsson, G. et al. (1994) J. Urol. 151, 446-448). Instillation of the vanilloid receptor agonists capsaicin or resiniferatoxin (RTX) into the bladder of patients with neurological deficits have been shown to improve symptoms of overactive bladder and to revert a previously positive IWT to negative in a number of patients (Geirsson, G. et al. (1995) J. Urol. 154, 1825-1829; Silva et al. (2000) Eur. Urol. 38, 444-452).

An excitatory bladder-to-bladder cooling reflex has also been identified in the α-chloralose anaesthetised cat with an intact neuraxis (Fall, M. et al. (1990) J. Physiol. 427, 281-300) mediated via a spinal pathway triggered by activation of unmyelinated C-fibre afferents in the bladder wall (Mazieres et al. (1998) J. Physiol. 513.2, 531-541; Jiang et al. (2002) J. Physiol. 543, 211-220). In contrast, normal micturition in response to bladder distension involves activation of myelinated (A-δ) mechanoreceptor afferents (Habler et al. (1993) J. Physiol. 463, 449-460) and a spinal-ponto-spinal reflex pathway (Holstege et al. (1986) J. Comp. Neurol. 250, 449-461). It has been proposed that the bladder cooling reflex originates from specific cold receptors since it is evoked at bladder volumes or pressures below threshold for bladder mechanoreceptors and the threshold temperature for the reflex in both cat and human is actually considerably higher than that required for activation of nociceptors (Fall et al. (1990) J. Physiol. 427, 281-300; Geirsson, G. (1993) J. Urol. 150, 427-430). In addition, menthol which has a selective potentiating effect on peripheral cold receptors (Hensel and Zotterman (1951) Acta Physiol. Scand. 24, 27-34) also sensitises the bladder cooling reflex towards higher temperatures (Geirsson, G. (1993) J. Urol. 150, 427-430; Lindstrom and Mazieres (1991) Acta Physiol. Scand. 141, 1-10). More recently, a cold- and menthol-sensitive receptor (CMR1) present in a subset of sensory neurones has been characterised and cloned (McKemy et al. (2002) Nature 416, 52-58).

In contrast to human and the α-chloralose anaesthetised cat, infusion of cold saline into the bladder of urethane-anaesthetised rats has been reported to elicit a bladder-to-external urethral sphincter reflex that results in an increased micturition pressure threshold (Cheng et al. (1997) Am. J. Physiol. 272, R1271-R1282). The rat cooling reflex is therefore not a suitable animal model for the evaluation of potential therapeutic agents for the treatment of incontinence.

However, it would be highly desirable for the pharmaceutical industry to have a well characterised animal model which showed an IWT response resembling the IWT in humans, since the bladder cooling reflex may be a biomarker of up-regulated C-fibre activation contributing to neuropathically-induced symptoms of overactive bladder in patients. New potential therapeutic agents for the treatment of incontinence could then be evaluated in an animal with the results indicative of the effect in human patients. Cats have been used for this purpose in some countries, but have the disadvantage that their use is restricted in some countries because of animal rights issues; additionally, it would be advantageous to have a model in smaller animals, which are easier and cheaper to keep. This would have the additional advantage of requiring much less material of the potential new therapeutic agent.

Surprisingly, we have found that the guinea pig, another rodent closely related to the rat, shows a response in the IWT which closely resembles the human response. Therefore, we can now use this guinea pig model for the evaluation of potential therapeutic agents for the treatment of incontinence, associated with overactive bladder, with a high probability that the results will be indicative of how a human patient will respond to the agent. As the guinea pig is small and easy to handle and keep, this is a significant advantage over the only other known animal model available for this purpose, which is the cat.

One aspect of the invention is therefore a method for evaluating potential therapeutic agents for the treatment of incontinence, preferably for the treatment of overactive bladder, comprising administering the potential therapeutic agent to a mammal, instilling cold liquid into the bladder of the mammal, and measuring the lowering of the threshold volume for bladder contractions compared to the normal cystometric capacity in the mammal, characterised in that the mammal is a guinea pig. Another aspect of the invention is the above method, wherein the potential therapeutic agent is administered to a guinea pig and the vehicle of the potential therapeutic agent is administered to a second guinea pig, followed by instilling cold liquid into the bladder of both guinea pigs and comparing the lowering of the threshold volume for bladder contractions compared to the normal cystometric capacity in both guinea pigs. Preferably the cold liquid will have a temperature of 0-15° C., more preferably a temperature of 0-10° C., even more preferably a temperature of 0-5° C., most preferably a temperature of 3° C.

Preferably the liquid to be instilled into the bladder will be water or saline solution.

Preferably, the compound will be instilled in the bladder, even more preferably, it will be administered intravenously, most preferably, it will be administered orally as pretreatment prior to anaesthetising the animal.

Another aspect of the invention is the above method, but comprising an additional step prior to the instillation of the cold liquid, that is instilling menthol solution into the bladder to sensitise the bladder to the cold liquid.

Another aspect of the invention is a method for screening for compounds for the treatment of incontinence, using the method mentioned above for many different compounds, and selecting the compounds which increase the threshold for bladder contractions to occur to the normal cystometric capacity, for further evaluation.

Another aspect of the invention is a method for preparing a pharmaceutical composition, comprising evaluating a potential therapeutic agent for the treatment of incontinence according to the method mentioned above, and admixing said compound with a pharmaceutically acceptable excipient.

For the avoidance of doubt, the term “compound” may refer to a chemical or biological agent, and includes, for example, antibodies, antibody fragments, other proteins, peptides, sugars, any organic or inorganic molecules. Compounds that may be used for screening include, but are not limited to, peptides such as, for example, soluble peptides, including but not limited to members of random peptide libraries; (see, e.g., Lam et al. (1991) Nature 354, 82-84; Houghten et al. (1991) Nature 354, 84-86), and combinatorial chemistry-derived molecular library made of D- and/or L-configuration amino acids, phosphopeptides (including, but not limited to, members of random or partially degenerate, directed phosphopeptide libraries; see, e.g., Songyang et al. (1993) Cell 72, 767-778), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules.

For orally bioavailable compounds, oral delivery will be preferred. However, other modes of delivery, including intravenous delivery and direct instillation into the bladder, are also envisaged.

Oral bioavailablity refers to the proportion of an orally administered drug that reaches the systemic circulation. The factors that determine oral bioavailability of a drug are dissolution, membrane permeability and hepatic clearance. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the compounds have a minimum solubility of 50 μg/ml. Solubility can be determined by standard procedures known in the art such as described in Lipinski C A et al.; Adv. Drug Deliv. Rev. 23(1-3), 3-25, 1997.

Membrane permeability refers to the passage of a compound through the cells of the GIT. Lipophilicity is a key property in predicting this and is determined by in vitro Log D_{7.4} measurements using organic solvents and buffer. Preferably the compounds have a Log D_{7.4} of −2 to +4, more preferably −1 to +3. The Log D can be determined by standard procedures known in the art such as described in Stopher, D and McClean, S; J. Pharm. Pharmacol. 42(2), 144, 1990.

Cell monolayer assays such as Caco2 add substantially to prediction of favourable membrane permeability in the presence of efflux transporters such as P-glycoprotein, so-called Caco2 flux. Preferably, the compounds have a Caco2 flux of greater than 2×10⁻⁶ cms⁻¹, more preferably greater than 5×10⁻⁶ cms⁻¹. The Caco2 flux value can be determined by standard procedures known in the art such as described in Artursson, P and Magnusson, C; J. Pharm. Sci, 79(7), 595-600, 1990.

Metabolic stability addresses the ability of the GIT to metabolise compounds during the absorption process or the liver to do so immediately post-absorption: the first pass effect. Assay systems such as microsomes, hepatocytes etc are predictive of metabolic lability. Preferably compounds show metabolic stability in the assay system that is commensurate with an hepatic extraction of less then 0.5. Examples of assay systems and data manipulation are described in Obach, R S; Curr. Opin. Drug Disc. Devel. 4(1), 36-44, 2001 and Shibata, Y et al.; Drug Met. Disp. 28(12), 1518-1523, 2000.

Because of the interplay of the above processes, further support that a drug will be orally bioavailable in humans can be gained by in vivo experiments in animals. Absolute bioavailability is determined in these studies by administering the compound separately or in mixtures by the oral route. For absolute determinations (% orally bioavailable) the intravenous route is also employed. Examples of the assessment of oral bioavailability in animals can be found in Ward, K W et al.; Drug Met. Disp. 29(1), 82-87, 2001; Berman, J et al.; J. Med. Chem. 40(6), 827-829, 1997 and Han K S and Lee, M G; Drug Met. Disp. 27(2), 221-226, 1999.

The compounds can be administered alone but will generally be administered in admixture with a suitable pharmaceutical excipient, diluent or carrier selected with regard to the intended route of administration and standard pharmaceutical practice.

For example, the compounds can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The compounds of the invention may also be administered as fast-dispersing or fast-dissolving dosage forms or in the form of a high energy dispersion or as coated particles. Suitable formulations may be in coated or uncoated form, as desired.

Such solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

The following formulation examples are illustrative only and are not intended to limit the scope of the invention. Active ingredient means a compound of the invention.

### FORMULATION 1

A tablet is prepared using the following ingredients:

Active ingredient (50 mg) is blended with cellulose (microcrystalline), silicon dioxide, stearic acid (fumed) and the mixture is compressed to form tablets.

### FORMULATION 2

An intravenous formulation may be prepared by combining active ingredient (100 mg) with isotonic saline (1000 ml)

The tablets are manufactured by a standard process, for example, direct compression or a wet or dry granulation process. The tablet cores may be coated with appropriate overcoats.

Solid compositions of a similar type may also be employed as fillers in gelatin or HPMC capsules. Preferred excipients in this regard include lactose, starch, a cellulose, milk sugar or high molecular weight polyethylene glycols. For aqueous suspensions and/or elixirs, the compounds may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds can also be administered parenterally, for example, intracavernousily, intravenously, intra-arterially, intraperitoneally, intrathecally, intraventricularly, intraurethrally, intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

For oral and parenteral administration to human patients, the daily dosage level of a compound will usually be from to 5 to 500 mg/kg (in single or divided doses).

Thus tablets or capsules may contain from 5 mg to 250 mg (for example 10 to 100 mg) of the compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will appreciate that the compounds may be taken as a single dose as needed or desired (i.e. prn). It is to be appreciated that all references herein to treatment include acute treatment (taken as required) and chronic treatment (longer term continuous treatment).

The compounds can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the compounds of the invention and a suitable powder base such as lactose or starch.

Aerosol or dry powder formulations are preferably arranged so that each metered dose or “puff” contains from 1 μg to 50 mg of a compound of the invention for delivery to the patient. The overall daily dose with an aerosol will be in the range of from 1 μg to 50 mg which may be administered in a single dose or, more usually, in divided doses throughout the day.

Alternatively, the compounds can be administered in the form of a suppository or pessary, or they may be applied topically in the form of a gel, hydrogel, lotion, solution, cream, ointment or dusting powder. The compounds of the invention may also be dermally or transderm ally administered, for example, by the use of a skin patch, depot or subcutaneous injection. They may also be administered by the pulmonary or rectal routes.

For application topically to the skin, the compounds can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in published international patent applications WO91/11172, WO94/02518 and WO98/55148.

Oral administration of the compounds is a preferred route, being the most convenient. In circumstances where the recipient suffers from a swallowing disorder or from impairment of drug absorption after oral administration, the drug may be administered parenterally, sublingually or buccally.

The invention will now be exemplified. The examples below are carried out using standard techniques, which are well-known and routinely used by those skilled in the art; the examples illustrate but do not limit the invention.

The following figures are referred to in the examples below:

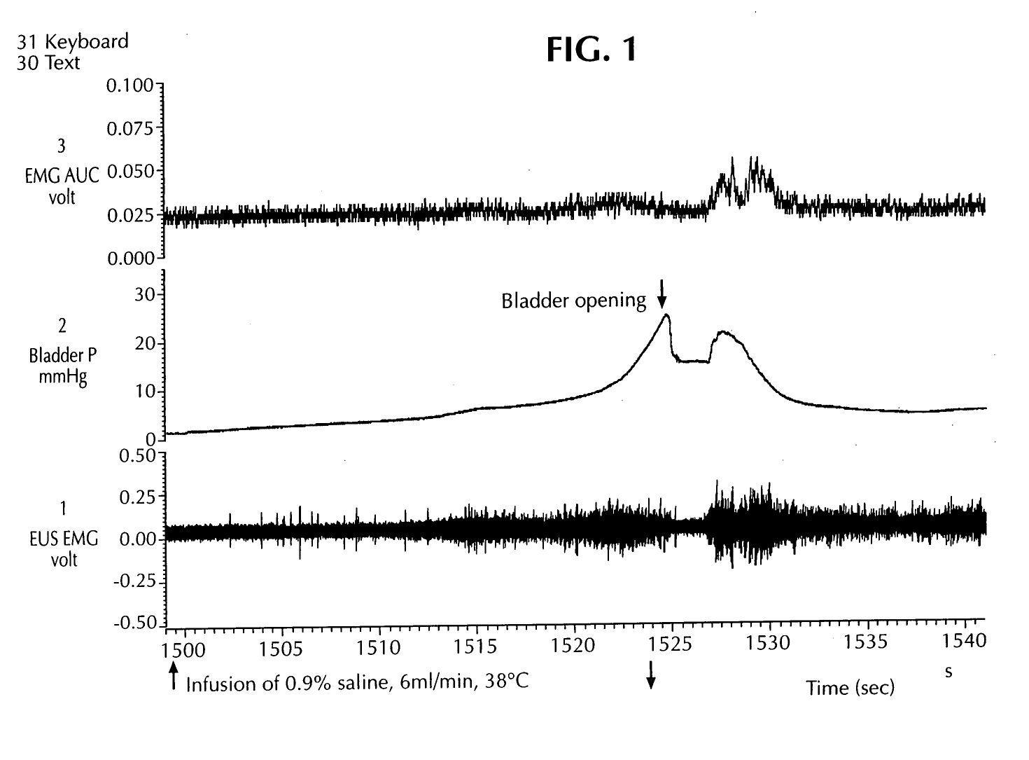

FIG. 1: Typical response to bladder infusion of 38° C. saline

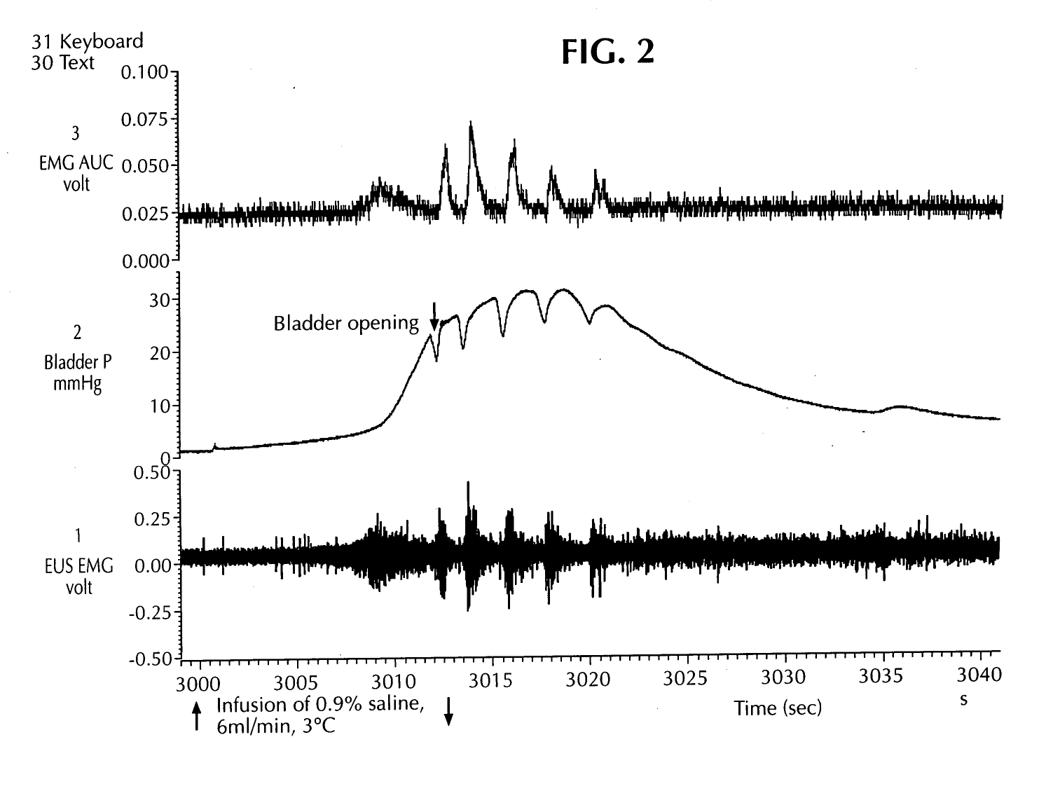

FIG. 2: Typical response to bladder infusion of 3° C. saline

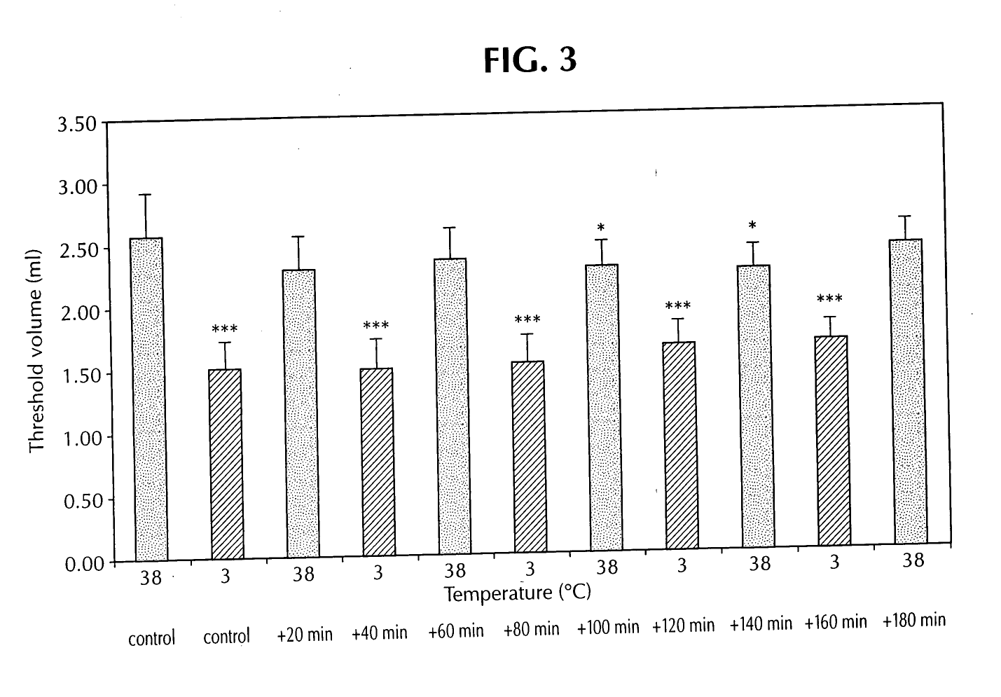

FIG. 3: Effect of repeated IWTs on micturition threshold volume in guinea-pig bladder

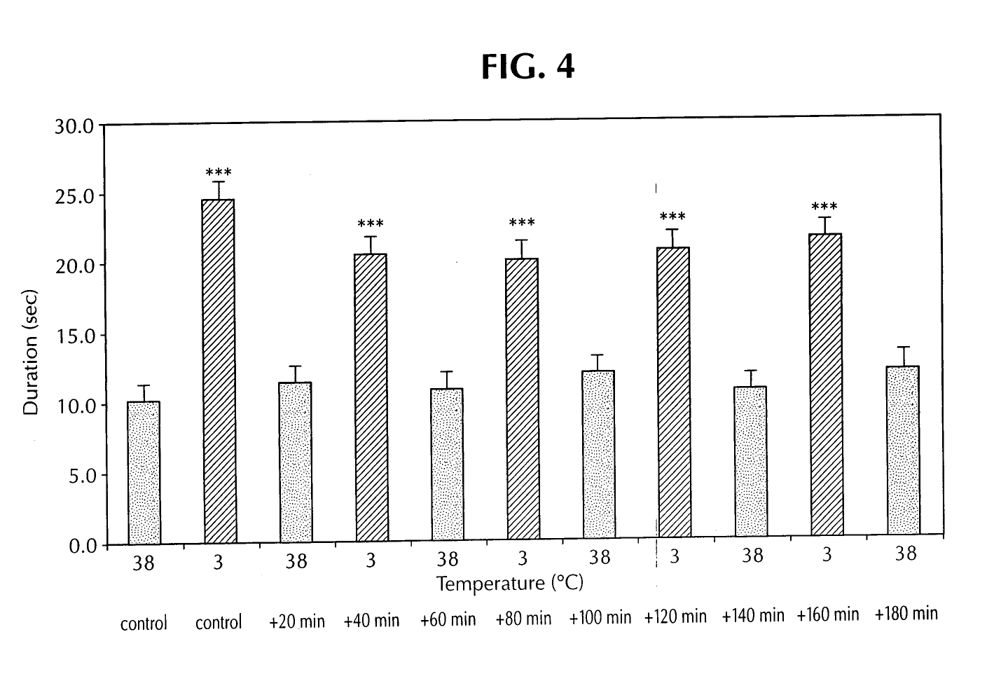

FIG. 4: Duration of pressure increase to repeated infusions of cold and warm saline in guinea-pig bladder

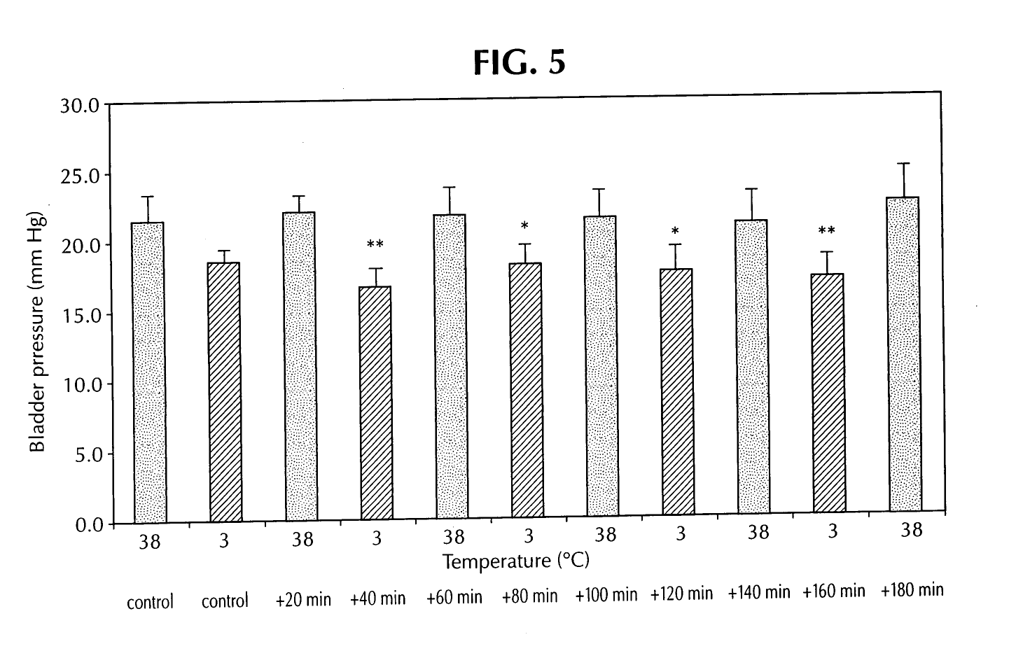

FIG. 5: Bladder opening pressure in response to repeated IWTs in guinea-pig bladder

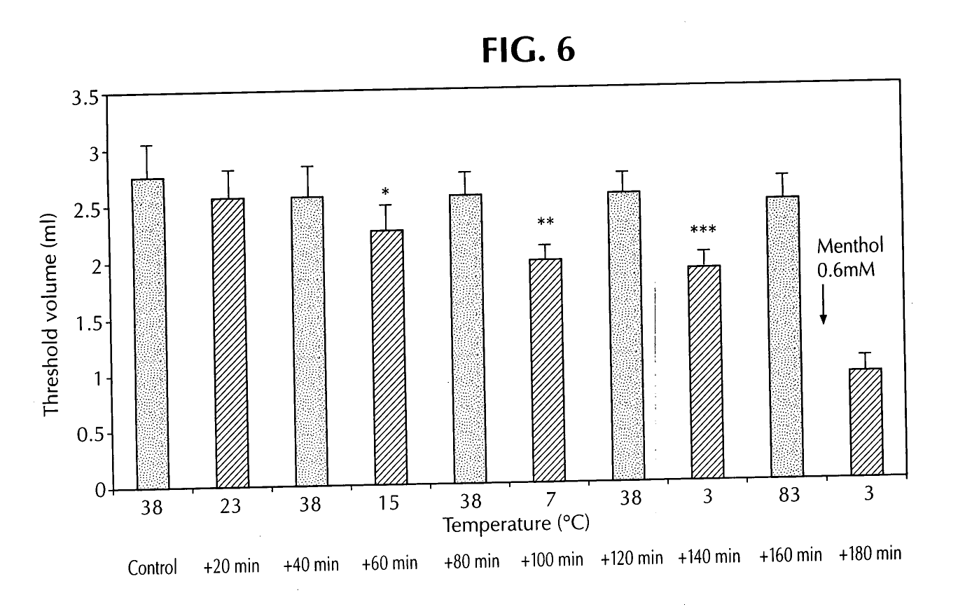

FIG. 6: Effects of infusate temperature and menthol on threshold volume for micturition in anaesthetised guinea-pig

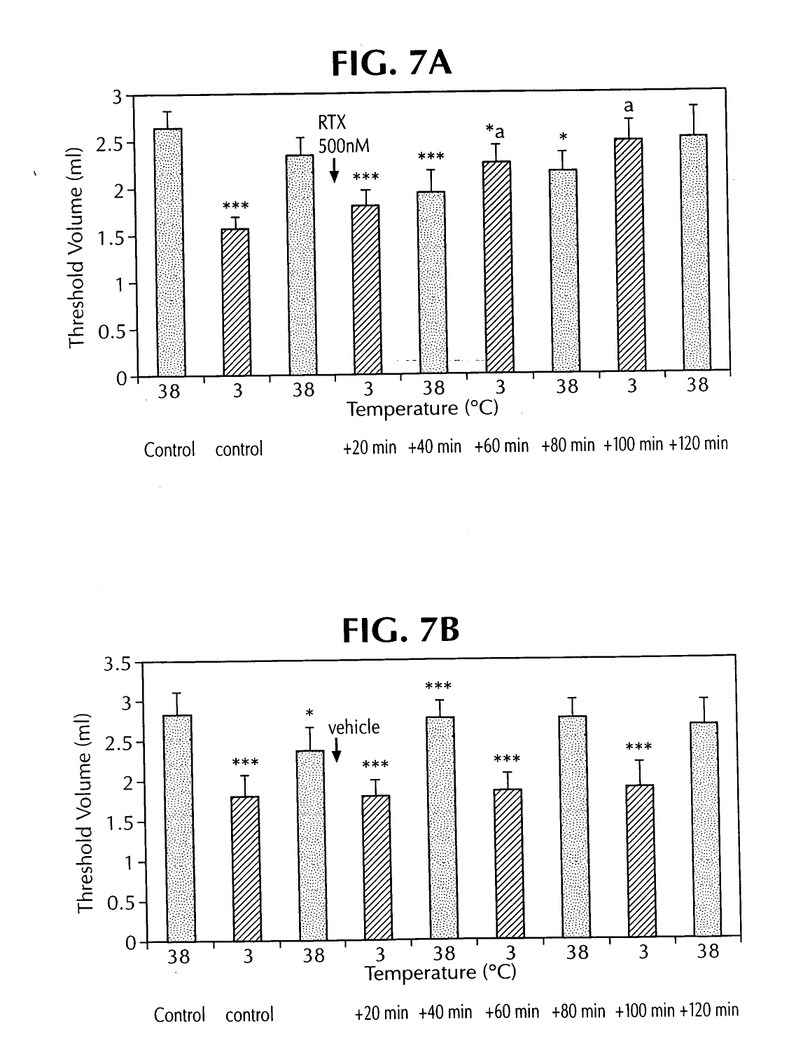

FIG. 7: RTX-induced desensitisation of the IWT response in guinea-pig bladder (a) as compared to vehicle-treated controls (b)

### EXAMPLE 1

### The IWT in Guinea Pig Bladder

Anaesthesia was induced in female Dunkin-Hartley guinea-pigs (326-416 g) by exposure to 4% halothane in oxygen. A surgical level of anaesthesia was then obtained by administration of 1.2 g/kg i.p. urethane followed by 40 mg/kg α-chloralose via a catheterised jugular vein. Supplementary doses of urethane (0.05 g/kg i.v.) were given if required. Rectal temperature was maintained between 36.5 and 37° C. via a Harvard homeothermic blanket control unit and additional heating lamp. A carotid artery was catheterised to monitor arterial blood pressure via a pressure transducer (Spectramed, Statham), the signal amplified and displayed on a Gould TA4000 chart recorder. The trachea was cannulated and the animals artificially respired using an Ugo-Basile rodent ventilator. A carotid arterial blood sample (0.3 ml) was analysed for blood gases using a GEM Premier 3000 analyser and the ventilation volume adjusted, with air intake enriched with O₂ if necessary, to set the level of blood PO₂>70 mmHg, PCO₂<40 mmHg and pH between 7.36 and 7.46.

The urinary bladder was exposed via a midline laparotomy and a double lumen catheter inserted into the lumen through an incision in the dome. The catheter was tied in place below the flanged tip of the outer tubing, followed by closure of the abdominal incision. The bladder catheter was constructed from a 3.5 cm length of tubing (ID 1.4 mm, OD 1.9 mm) inside which 2 catheters were sealed. One catheter connected to a pressure transducer (Spectramed, Statham) was used to record bladder pressure and to withdraw residual volume via a syringe on a plastic stopcock attached to the transducer. The second catheter was used for infusion of 0.9% saline and consisted of a 5 cm length of tubing (ID 0.5 mm, OD 1.0 mm) connected to a 11 cm length of silicone tubing (ID 0.8 mm, OD 4 mm) inserted into coiled glass tubing within a water jacket through which water circulated at various temperatures. The thermal dead space between the tip of the catheter in the bladder and the glass coil was 70 μl. The other end of the coiled glass tubing was connected to a syringe infusion pump (Kd Scientific). Thermostatically controlled cooling of the infusate below room temperature was achieved by means of a Grant C1G refrigerated immersion cooler in conjunction with a Grant W6 circulating water bath whilst warming of the infusate was achieved by switching to water circulating at 38° C. from a separate Grant W6 water bath. Two braided stainless steel wire electrodes, insulated except for approximately 5 mm at their tips were inserted percutaneously on either side of the external urethral opening within the tips of 23G hypodermic needles which were then withdrawn leaving the electrodes positioned on either side of the external urethral sphincter (EUS). The amplified EUS electromyogram (EUS EMG), the integrated EUS EMG and bladder pressure signals were recorded using Spike2 software (Cambridge Electronic Design). Saline at room temperature was infused into the bladder at 150 μl/min to elicit micturition reflexes and associated EUS EMG activity to confirm correct positioning of the electrodes. The infusion of saline was then stopped and a stabilisation period of at least 1 hr allowed before starting the experimental protocol.

Micturition reflexes (MRs) were elicited by the rapid infusion of 0.9% saline into the bladder at 6 ml/min. Infusions were stopped at the start of voiding and the residual volume withdrawn after 60 sec. In every experiment, warm (38° C.) saline was initially infused at 10 min intervals until usually 3 reproducible responses were obtained, the mean value of which gave the control 38° C. response for that animal.

The threshold volume (TV) for each MR was defined as the volume of saline required to elicit bladder opening seen as a peak in bladder pressure (bladder opening pressure) on the Spike2 recording that was followed by a sudden drop in pressure and the appearance of the first voided fluid. The maximum bladder pressure during each MR was also measured using the Spike2 software, a data acquisition and analysis system produced by Cambridge Electronic Design, Science Park, Milton Road, Cambridge, England.

Data is presented in graphical format as mean±s.e.m. of the raw data. Analysis of data was mainly by ANOVA, but ANOCOVA was also used, both being standard statistical analyses known to the skilled person. Values of P<0.05 were considered significant.

Following control MRs at 38° C., a micturition reflex to a single infusion of cold (3° C.) saline was obtained (control) and then alternate warm and cold saline infusions at 20 min intervals over a 3 hr period.

FIG. 1 shows the typical response to infusion of saline at 38° C., whereas FIG. 2 shows the typical response to infusion of saline at 3° C. FIG. 3 shows the effect of repeated IWTs on micturition threshold volume in guinea-pig bladder, measured in 20 minute intervals alternating between infusions with saline at 38° C. and 3° C. (*: p<0.05; ***: p<0.001 compared with mean 38° C. control; n=6). FIG. 4 shows the duration of pressure increase to repeated infusions of cold and warm saline in guinea-pig bladder (***: p<0.001 compared with mean 38° C. control, n=5 or 6).

The micturition threshold volume of 1.52 ml for the 3° C. control saline infusion into the bladder was significantly lower than the threshold volume of 2.58 ml for the 38° C. control infusion (diff. −1.06, 95% C.I. −1.34, −0.78, p<0.001), equivalent to 60% (range 46-80%) of bladder capacity at 38° C. This significant reduction in threshold volume was repeatable during several subsequent infusions at 3° C. (FIG. 3). The duration of the rise in bladder pressure was doubled by cold saline (FIGS. 1, 2, and 4) and showed oscillations associated with bursting activity in the EUS EMG (FIG. 2). Bladder opening pressure was slightly reduced in response to infusion of cold compared with warm saline, statistically significant with repeated infusions (FIG. 5; *: p<0.05; **: p<0.01 compared with mean 38° C. control). Bladder pressure continued to rise after the bladder opened following cold saline infusion in some animals but maximum bladder pressure was only significantly increased for the first 3° C. response (25.5 mmHg) compared with the 38° C. control (21.9 mmHg) (diff. 3.7, 95% C.I. 0.1, 7.3, p<0.05), a mean increase of 16.4%.

The rapid infusion of cold (3° C.) saline into the bladder of anaesthetised guinea-pigs reduced the threshold volume required to elicit a micturition reflex to 60% (range 46-80%) of the threshold volume at 38° C. Similarly, rapid infusion (300 ml/min) of ice-cold water into the bladder of patients with neurological disorders results in a reduction in threshold volume, Geirsson et al. ((1994) Br. J. Urol. 73, 498-503) reporting a median threshold volume of 32% (range 9-60) of the cystometric capacity. Fall et al. ((1990) J. Physiol. 427, 281-300) described a bladder-bladder cooling reflex in the anaesthetised cat with a reduction in threshold volume to elicit reflex detrusor contractions on rapid (0.3-2 ml s⁻¹) bladder infusion of cold compared with body-warm saline.

In contrast, in urethane-anaesthetised rats, using a slow continuous infusion (0.052 ml/min) cystometry model, no significant reduction in the interval between micturition reflexes during cold saline infusion (6-8° C.) compared with infusion at room temperature (25-26° C.) was found (Cheng et al., (1997) Am. J. Physiol. 272, R1271-R1282). Contrary to the reduction in threshold pressure in human patients and cats, and the reduction in bladder opening pressure in guinea-pigs, in rats cold saline elicited a small increase in baseline bladder pressure and a marked increase in the pressure threshold for inducing a micturition reflex (mean, 87% increase). In addition, cold saline infusion in the rat increased bladder contraction amplitude by ˜100% in contrast to the mean increase in maximum bladder pressure of 16% in the guinea-pig and lack of significant difference in maximum cystometric micturition pressure and maximum detrusor pressure during a positive IWT in patients (Geirsson G. et al. (1994) Br. J. Urol. 73, 498-503). Pudendal nerve section or neuromuscular blockade in the rat eliminated or markedly reduced the effect of cold stimulation and accompanying increase in EUS EMG activity as did subcutaneous pre-treatment with capsaicin. It was concluded that reflex activation of the EUS via capsaicin-sensitive bladder afferents stimulated by cold produced an increase in urethral outlet resistance, thus impeding voiding, and resulting in the observed increase in pressure amplitude, but a facilitatory effect of bladder cold receptors on reflex bladder contractions, as postulated and consistent with the observed effects in human patients, cats and guinea pigs, was not prominent in the rat.

### EXAMPLE 2

### Temperature Threshold and the Effect of Menthol for the IWT in Guinea-Pig Bladder

Animals were prepared as described in Example 1 above, and the protocol for control micturition reflexes at 38° C. in guinea-pig bladder as described in Example 1 was applied for this example as well. The data were analysed as described in Example 1.

Following control micturition reflexes at 38° C., MRs to infusions of saline were obtained at 20 min intervals, alternating infusions at lower temperatures of 23° C. (room temperature), 15° C., 7° C. and 3° C. with infusions at 38° C. After the last 38° C. infusion, 0.6 mM menthol was infused for 5 min at 0.3 ml/min directly into the bladder at room temperature. Residual fluid was then withdrawn from the bladder followed by rinsing with saline at room temperature and withdrawal of residual volume. Treatment with menthol was carried out by direct infusion into the bladder catheter which was disconnected from the glass coil thus bypassing the large dead space volume in the coil. Saline infusion at 3° C. was re-tested 10 to 12 min after completing the menthol infusion (20 min after the preceding 38° C. infusion).

Graded reductions in temperature from 23° C. to 3° C. resulted in progressive reductions in TV, statistically significant at 15° C. (FIG. 6; *: p<0.05, **: p<0.01; ***: p<0.001 compared with mean 38° C. control (n=4); a: p<0.001 compared with 3° C. before menthol). The TV of 1.90 ml at 3° C. was subsequently further reduced to 0.95 ml following treatment of the bladder with 0.6 mM menthol (diff. −0.95, 95% C.I. −1.33, −0.57, p<0.001).

Although difficult to compare, the reductions in TV observed in the guinea pig with decrease of temperature of the infused saline seems to be roughly similar to those observed in human patients, whereas a slightly higher dynamic threshold temperature of 30-32° C. was reported for the anaesthetised cat (Fall et al (1990) J. Physiol. 427, 281-300).

The significant further reduction of the threshold volume to 3° C. in the anaesthetised guinea-pig with menthol treatment was in line with results from human patients and cats. Potentiation of the bladder cooling reflex by a shift of the threshold temperature towards higher temperatures was demonstrated with 0.5 mM menthol in patients (Geirsson (1993) J. Urol. 150, 427-430) and 0.6 mM menthol in cats (Lindstrom and Mazieres (1991) Acta Physiol. Scand. 141, 1-10).

### EXAMPLE 3

### Effect of Resiniferatoxin (RTX) Treatment as Compared to Vehicle-Treated Controls

Animals were prepared as described in Example 1 above, and the protocol for control micturition reflexes at 38° C. in guinea-pig bladder as described in Example 1 was applied for this example as well. The data were analysed as described in Example 1.

Following control MRs at 38° C., a micturition reflex to a single infusion of cold (3° C.) saline was obtained (control) and then a further MR to infusion at 38° C. after a 20 min interval. Animals were subsequently treated with either 500 nM RTX or its vehicle (0.05% alcohol, 0.05% Tween 80 in 0.9% saline) infused into the bladder at room temperature at 6 ml/min until voiding followed by continuous infusion at 0.3 ml/min for 30 min. Residual fluid was then withdrawn from the bladder followed by rinsing with saline at room temperature and withdrawal of residual volume. Treatment with RTX or vehicle was carried out by direct infusion into the bladder catheter which was disconnected from the glass coil thus bypassing the large dead space volume in the coil. Alternate infusions of cold (3° C.) and warm (38° C.) saline were retested at 20 min intervals from 20 min to 120 min after terminating the RTX or vehicle infusion.

Desensitisation of the 3° C. induced reduction in TV was shown following treatment with 500 nM RTX (FIG. 7(a); *: p<0.05, ***: p<0.001 compared with mean 38° C. control; a: p<0.001 compared with 3° C. control). At 100 min post RTX infusion, the TV to 3° C. was not significantly different from the TV for the 38° C. control response before RTX (diff. −0.16, 95% C.I. −0.51, 0.20, p>0.05). In contrast, no desensitisation was seen in the control group. At 100 min post vehicle infusion, (FIG. 7(b); *: p<0.05; ***: p<0.001), the TV to 3° C. saline infusion was still significantly different from the 38° C. control (diff. −0.95, 95% C.I. −1.37, −0.53, p<0.001).

This result in guinea-pigs resembles results obtained in human patients, again indicating that the guinea pig is a suitable animal model. Silva et al. ((2000) Eur. Urol. 38, 444-452) showed acute and/or chronic reversal of a previously positive IWT following bladder treatment with RTX in patients with detrusor hyperreflexia. The IWT was positive in 13 patients at baseline. Immediately after instillation of 50 or 100 nM RTX, the IWT became negative in 4 patients and in an additional 2 at 14 days. At 90 days post treatment, the test was negative in 8 cases.

### EXAMPLE 4

### Evaluation of Potential Therapeutic Agents for the Treatment of Incontinence

The experiment is carried out as described in Example 3 above, except that instead of RTX, the compound to be evaluated is infused into the bladder. The skilled person will be aware that the compound can also be administered, for example, orally (by pre-treatment) or intravenously. The results for a compound that is beneficial for the treatment of incontinence will mirror those obtained with RTX.

1. A method for evaluating potential therapeutic agents for the treatment of incontinence, comprising administering the potential therapeutic agent to a mammal, instilling cold liquid into the bladder of the mammal, and measuring the lowering of the threshold volume for bladder contractions compared to the normal cystometric capacity, characterised in that the mammal is a guinea pig.

2. The method of claim 1, wherein the potential therapeutic agent is administered to a guinea pig and the vehicle of the potential therapeutic agent is administered to a second guinea pig, followed by instilling cold liquid into the bladder of both guinea pigs and comparing the lowering of the threshold volume for bladder contractions compared to the normal cystometric capacity in both guinea pigs.

3. The method of claim 1, wherein the cold liquid instilled into the bladder has a temperature between 0 and 15° C.

4. The method of claim 1, wherein the cold liquid instilled into the bladder has a temperature between 0 and 10° C.

5. The method of claim 1, wherein the cold liquid instilled into the bladder has a temperature between 0 and 5° C.

6. The method of claim 1, wherein the liquid is water.

7. The method of claim 1, wherein the liquid is saline.

8. The method of claim 1, wherein the potential therapeutic agent is administered by intravenous infusion.

9. The method of claim 1, wherein the potential therapeutic agent is administered orally by pretreatment.

10. A method for screening for compounds for the treatment of incontinence, using the method of claim 1 for many different agents.

11. A method for preparing a pharmaceutical composition, comprising evaluating a potential therapeutic agent for the treatment of incontinence according to the method of claim 1, and admixing said agent with a pharmaceutically acceptable excipient.
